(19) United States
(12) Patent Application Publication
(10) Pub. No.: US 2022/0204897 A1
(43) Pub. Date: Jun. 30, 2022

(54) LIGHT AND TEMPERATURE DEVICE FOR OPTIMZING THE PRODUCTION OF BIOMASS IN BIOREACTORS

(71) Applicant: Rhenac Greentec AG, Hennef (DE)

(72) Inventor: Horst Theisen, Bonn (DE)

(21) Appl. No.: 17/607,461

(22) PCT Filed: Apr. 28, 2020
(86) PCT No.: PCT/EP2020/061787

§ 371 Date: Oct. 29, 2021

(30) Foreign Application Priority Data
Apr. 29, 2019 (DE) 10 2019 110 989.2

(51) Int. Cl. C12M 1/00; C12M 1/02; C12M 3/00

(52) U.S. Cl. CPC C12M 21/02; C12M 31/02; C12M 41/06; C12M 41/18; C12M 23/48; C12M 31/10

(57) ABSTRACT

The invention relate to a light and temperature device for increasing and optimizing the production of biomass in bioreactors, said device comprising an LED light arrangement with LED light arrays and cooling fins facing away from the LED light arrays. The light and temperature device further comprising at least one fastening means for removably fastening the light arrangement to a transparent incubation vessel the LED light arrangement being oriented in relation to the incubation vessel such that light radiation emitted by the LED light arrangement in directed towards the incubation vessel.

### FIELD OF INVENTION

The invention relates to a light and temperature device for increasing and optimizing the production of biomass in bioreactors. In particular, the invention relates to the provision of a light and temperature sleeve for the production of microorganisms such as microalgae in photobioreactors. In such photobioreactors, light, CO₂, nutrients as well as water and salts are mainly used to propagate and/or cultivate microorganisms. These microorganisms or their components are mainly used for the production of oils, fuels, food, food additives, enzymes and proteins, pharmacological agents, chemical products, plastics, or are used in aquacultures, animal feed, environmental technology and for cosmetics and care.

### BACKGROUND OF THE INVENTION

A bioreactor is a unit for the production of microorganisms outside their natural environment and within an artificial technical environment. The prefix “photo” describes the characteristic of the bioreactor to serve for the cultivation of phototrophs, i.e. organisms using light for their own energy production. These organisms use the process of photosynthesis to build up their own biomass from light and CO₂. These organisms include plants, mosses, macroalgae, microalgae, cyanobacteria and purple bacteria. The central objective of a photobioreactor is the controlled provision of a habitat that provides the optimal living conditions for the organism in question. Thus, a photobioreactor enables significantly higher growth rates and purities than would be the case in a natural or nature-like environment.

Thus, photobioreactors are systems for the cultivation of mainly various eukaryotic organisms that live in water and perform photosynthesis, such as microalgae. Such systems are known from the prior art. For example, a widely used system for the cultivation of microorganisms is a tube circulation system consisting of tubes that form a quasi-infinite loop by changing direction in repetitive loops. In these tubes, cultured microorganisms are kept in circulation and in motion by means of pump systems. In the closed loop, the microorganisms grow by feeding carbon dioxide and nutrients that are introduced into the system.

The growing phototrophic microorganisms also need light to perform photosynthesis and to grow. Light is a decisive factor for the optimal productivity of the entire phototrophic microorganisms or the entire system. Usually, in order to keep the light factor at the required light intensity throughout the year, supplementary light is also used. For this purpose, light sources such as sodium vapor lamps, metal halide lamps are placed around the entire tube system or above this system as supplementary lighting. However, this supplementary lighting causes additional heating of the tube photobioreactors, which has proven to be unfavorable for phototrophic microorganism culture development; the microalgae cultures tend to adhere to the glass tube surfaces that are heating up.

Another disadvantage of such a system is that the light reaches the tubes only in parts, because the tubes cast shadows on each other. This effect increases when the phototrophic microorganism cultures multiply in the circulating culture fluid and the fluid becomes increasingly green due to the higher chlorophyll content. If this effect occurs, only a few light rays reach the inside of the tubes, hereby, the effective amount of radiation that can participate in photosynthesis decreases and thus, also the yield of usable substances in the biomass.

Therefore, the object of the invention is to overcome the disadvantages of the previously described photobioreactors, in particular, those of the tube photobioreactors. Here, the aim is to improve the culture condition for the growth of phototrophic microorganisms or other biomass producers, in particular, by optimizing the light availability within such a photobioreactor.

Another object of the invention is to adjust the temperature conditions as optimal as possible for the phototrophic microorganisms.

The objects are solved by a light and temperature device according to the invention for optimizing the production of biomass in bioreactors according to independent claim 1. Advantageous further developments and preferred embodiments are specified in the dependent claims.

### SUMMARY OF THE INVENTION

According to the invention, a light and temperature device for optimizing the production of biomass in bioreactors, in particular, photobioreactors is provided, wherein here, the device comprises an LED light arrangement with LED light arrays and cooling fins, as well as at least one fastening means for detachable attachment of the light arrangement to a light-transmissive incubation container. Thereby, the LED light arrangement is arranged relative to the incubation container in such a way that a light radiation emanating from the LED light arrangement is directed towards the incubation container. Thereby, the cooling fins of the light arrangement are arranged such that they are facing away from the LED light arrays. Advantageously, the distance between the light array and the incubation container is kept as small as possible. This has the advantage that the light radiation is introduced into the incubation container with virtually no losses. Thus, shadowing by other adjacent incubation vessels is avoided and optimal light delivery to each individual incubation vessel of a composite photobioreactor can be achieved. Due to the controlled temperature conditions provided by the light and temperature device according to the invention, the incubation systems, e.g. tube systems, may also be changed from glass to plastic, since the expansion of the (tube) systems can be controlled by the constant temperature. This additionally reduces the cost of manufacturing the bioreactor systems. The light and temperature device according to the invention enables an increase of the dry biomass from currently about 10-15 grams/liter of circulating liquid to about 25-30 grams/liter. The invention would allow for the (tubular) bioreactors being designed with larger (tubular) diameters, significantly improving plant productivity per unit area.

The arrangement of the cooling fins behind the LED light arrays, with these facing away from the LED light arrays, has the advantage that this allows the waste heat from the LED light arrays to be dissipated away from the incubation vessel. This in turn has the consequence that the incubation container and thus the microorganisms contained therein are not additionally heated and unfavorable culture conditions are resulting for the microorganisms.

The re-releasable attachment of the light arrangement by at least one clamp has the advantage that the light and temperature device according to the invention can be adapted to the scaling of a photobioreactor system simply, quickly and as required.

Here, the at least one fastening means of the light and temperature device according to the invention may also be built up from different components. The fastening means itself may represent any kind of fastening means, as long as it is suitable for the releasable attachment of the light and temperature device according to the invention to a light-transmitting incubation container. Where appropriate, it may be advantageous to use more than one fastening means to attach multiple LED light arrangements to a light-transmissive incubation container or to increase the stability of the overall light and temperature device that is attached to the incubation container. Further, the fastening means may also serve to accommodate other optional elements such as measurement and/or monitoring devices. According to an advantageous embodiment of the light and temperature device according to the invention, the fastening means is a clamp.

Within the meaning of the invention, microorganisms are understood to be plants, mosses, macroalgae, cyanobacteria, purple bacteria, plankton and, in particular, microalgae. Preferably, the light and temperature device according to the invention is used for the cultivation of microalgae and is designed for this purpose.

Within the meaning of the invention, a bioreactor system or a photobioreactor system is understood to be a system built up from various components, for example, tubes, plates, films or other components suitable for cultivating microorganisms. In addition, such a photobioreactor system includes pumps, inlet and outlet valves for nutrients, CO₂, salts, water, etc., and fastening means for suspending or connecting the individual components.

Within the meaning of the invention, an incubation vessel is understood to be a component of a bioreactor system that is used primarily to propagate and/or cultivate microorganisms.

Such an incubation container may have various geometric shapes. Tubes, plates, films and/or tubes have proven to be particularly advantageous. When phototrophic microorganisms are used, these are naturally made of a light-transmissive material so that the microorganisms can carry out photosynthesis.

Advantageously, bioreactors of the tubular photobioreactor, fir tree photobioreactor, plate photobioreactor or foil photobioreactor design have shown to be effective. The light and temperature device according to the present invention may be applied to these as well as to all common photobioreactors.

According to an advantageous embodiment of the light and temperature device according to the invention, this is in the form of a sleeve. That is, the light and temperature device may be arranged or installed around an incubation container (e.g., arrangement around the tube lengths). This light and temperature device, designed as a sleeve and equipped with LED light arrays, provides the best possible light supply for the microorganism cultures. According to a further advantageous embodiment of the light and temperature device according to the invention, such a sleeve has further components such as casing or jacket parts in order to arrange the individual components of the sleeve around an incubation container, e.g. a tube, and to fix them by means of suitable fastening means (e.g. a clamp).

According to a further advantageous embodiment of the light and temperature device according to the invention, this comprises at least one channel for connecting cooling water or cooling liquid. In this case, this channel passes through the LED light arrangement and thus, may be used for active cooling of the LEDs. This has the advantage that, in addition to passive cooling by means of the cooling fins, active cooling may be implemented, for example, on hot days or more intensive light irradiation, and thus an optimum temperature level may be created for the propagation of the microorganism culture. Likewise, by controlled coolant circulation in the LED light arrangement, a desired temperature input may be created in the incubation container (e.g., tube system), so that cultivation may be performed with the same climate year-round and regardless of location.

According to a further advantageous embodiment of the invention, the device has suitable connections for supply and discharge of water or coolant. According to a further advantageous embodiment of the light and temperature device according to the invention, the device comprises at least one electrical interface for connecting measuring and/or monitoring devices. Such measuring and/or monitoring devices may include, for example, various cameras, such as CCD cameras, temperature sensors, spectrometers, viscometers and fluorescence and chlorophyll measuring devices.

Such measuring and/or monitoring devices may be designed to measure fluorescence, luminescence, radiation, fluidity, viscosity, color changes and/or chlorophyll content.

The advantage of attaching such measuring and/or monitoring devices to the light and/or temperature device according to the invention is that they are suitable for determining or detecting, for example, the optimum use of light, the harvesting time of the microorganism culture and/or, if necessary, possible contaminations of the culture. Likewise, a temperature sensor may be used, for example, to actively regulate the temperature, for example, via cooling water or cooling liquid, which may then be pumped into the channels of the LED light arrangement.

According to a further embodiment of the light and temperature device according to the invention, it has insulation between and/or to the side of the LED light arrangement and the incubation container. The insulation is attached in such a way that the light irradiation of the LED light arrangement onto the incubation container is not influenced thereby. Such insulation comprises, for example, rubber, plastic, polyurethanes (PUR) or polyisocyanurate rigid foam (PIR).

According to a further advantageous embodiment of the light and temperature device according to the invention, the fastening means may also comprise an insulation or a special insulation is held around the incubation container by means of a fastening means.

Likewise, insulation may also completely or partially encase or cover the entire incubation container—with the exception of the LED light unit. The insulation variants have the advantage that, for example, the incubation container is not damaged or scratched before possibly being damaged by metallic or hard components of the light and temperature device according to the invention. A further advantage of such insulation, for example, if it is made of rubber or the like, is that a certain slip resistance is also achieved in this case, which prevents the light and temperature device according to the invention, in particular, the LED light arrangement, from turning relative to the incubation container.

According to a further embodiment of the light and temperature device according to the invention, this may comprise one or more LED light arrangements which are arranged continuously about a longitudinal axis of an incubation container. In the case of a mainly circular cross-section of the incubation container, these LED light arrangements may be arranged at intervals of 1-360 degrees around the incubation container.

According to another advantageous embodiment of the light and temperature device according to the invention, the LED light arrangements are arranged continuously around the longitudinal axis of an incubation container at intervals of 180 degrees or of 135 degrees or preferably of 90 degrees.

According to a further advantageous embodiment of the light and temperature device according to the invention, the LED light arrangements are configured to emit a fixed light spectrum, also referred to as SMD (surface-mounted devices) design, or to emit a variable light spectrum. In the case of a variable light spectrum, which is also referred to as a multichannel design, each light color receives a separate channel for controlling 0-100% light output. The spectrum may then be controlled variably, according to demand.

According to a further embodiment of the light and temperature device according to the invention, it is designed in such a way that there is the smallest possible distance between the LED light arrangement or the LED light arrays and the incubation container in terms of construction. This constructional variant has the advantage that the energy input for the exposure of the microorganism culture may be considerably reduced as a result.

According to a further advantageous embodiment of the light and temperature device according to the invention, the latter is designed for detachable attachment to a tubular, cylindrical, cuboidal, prismatic, pentahedral, hexahedral, heptahedral, octahedral or otherwise geometrically configured elongated incubation container.

According to another advantageous embodiment of the light and temperature device according to the invention, the device is formed of materials suitable for passive cooling. For example, components of the light and temperature device have sections of aluminum or other metals or materials suitable for cooling. In particular, the cooling fins of the LED light arrangement are made of such a material.

### BRIEF DESCRIPTION OF THE DRAWINGS

The invention is described in further detail below with reference to specific embodiments and figures in which:

FIG. 1 shows a schematic three-dimensional illustration of the light and temperature device according to the invention in accordance with an advantageous embodiment in the attached state around an incubation container;

FIG. 2 shows a schematic illustration of a cross-section of the light and temperature device according to the invention shown in FIG. 1;

FIG. 3 shows an enlarged schematic illustration of the LED light arrangement, which is attached to the incubation container according to the section X of FIG. 2;

FIG. 4 shows a schematic side view of the light and temperature device according to the invention as shown in FIG. 1;

FIG. 5 shows a schematic top view of the light and temperature device according to the invention as shown in FIG. 1.

### DESCRIPTION OF THE PREFERRED EMBODIMENTS

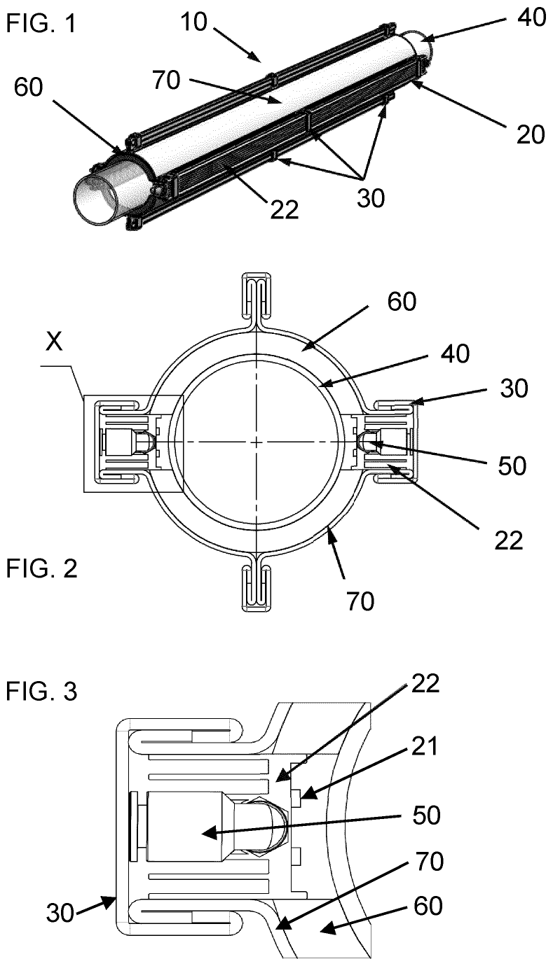

The invention is described below with reference to preferred exemplary embodiments and associated figures. It is noted that the figures here are purely schematic and do not allow any conclusions to be drawn as to the exact scale. The figures and the accompanying description merely serve to illustrate the principle of the invention, but these do not limit the invention in any way. In FIG. 1, a preferred embodiment of the light and temperature device 10 according to the invention is illustrated in a schematic three-dimensional illustration. Here, the light and temperature device 10 according to the invention is already in the assembled state on an opaque incubation container 40. Here, the incubation container 40 is a Plexiglas tube which may be assembled via deflection modules with other Plexiglas tubes to form a so-called tube photobioreactor. In these particular embodiments, the light and temperature device 10 according to the invention has two opposing LED light arrangements 20. Further, in this embodiment, the light and temperature device 10 according to the invention is in the form of a sleeve. The latter is formed around the incubation container by means of jacket components, in this case quarter shells 70, and clamps provided for this purpose as fastening means.

FIG. 2 shows the embodiment of FIG. 1 in a schematic cross-sectional view. The two LED light arrangements 20 can be seen, as well as the quarter shells 70. The quarter shells 70 also secure an insulating material 60 around the incubation container 40 by means of the clamps 30.

FIG. 3 shows, in an enlarged portion, a schematic cross-section of the LED light arrangement 20 of FIG. 2. Here, the LED light arrangement 20 can be seen having cooling fins 22 as well as LED light arrays 21. Further, a connection for water or cooling liquid is visible, which further opens into a channel 50 that extends through the LED light arrangement 20, in this case through the cooling fin profile 22. Also visible is a special clamp 30 for fixing the cooling profile 22 to the incubation container 40.

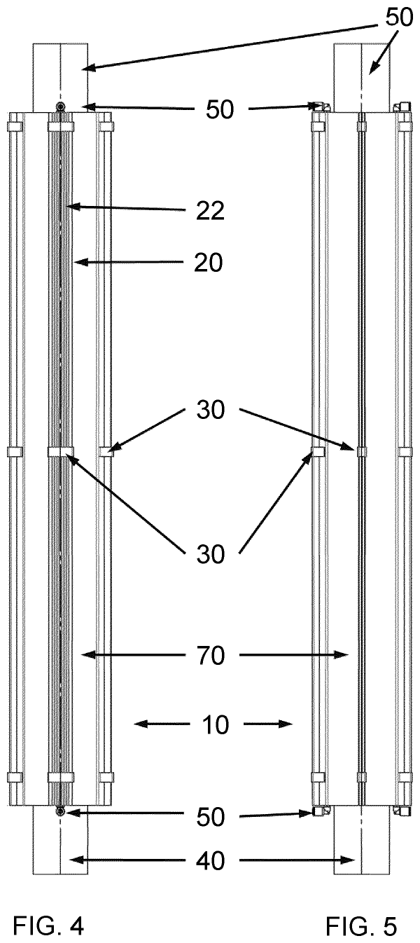

The particular embodiment of the light and temperature device 10 according to the invention as shown in FIG. 1 is shown in FIG. 5 in plan view and in FIG. 4 in side view.

This particular embodiment of the light and temperature device 10 according to the invention described in FIGS. 1-5 is particularly suitable for conception in tubular photobioreactors. Plexiglas tubes, for example, are suitable as incubation containers 40 for this purpose. In the particular embodiment, these tubes 40 are provided with the light and temperature device 10 according to the invention, which has the shape of a sleeve.

This embodiment is also referred to as a light sleeve. The light sleeves thereby may be placed around the tubes, wherein they are detachably attached again by means of quarter shells 70 and special clamps 30. The light sleeves contain LED light arrangements 20 which can couple light into the tubes directly, on one side or, as in this case, on two sides offset by 180 degrees. Thereby, the light sleeves are preferably made of aluminum or other materials suitable for cooling. In this embodiment, the LED light arrangements 20 have cooling fins 22 on the outside of the light arrangement 20 to allow the waste heat generated by the LEDs to be dissipated to the outside. In addition, the LED light arrangements 20 have cooling channels 50 with connections into which cooling liquid or water may be introduced. The temperature of the cooling liquid may be controlled via a connection to refrigeration systems, heat exchangers or the like. This allows for adjustable and controlled temperatures at the tubes, which has a positive effect on microorganism culture production. The light arrangements 20 equipped with LED light arrays 21 provide the best possible light delivery for the microorganism cultures. In this regard, the LED light arrangement 20 may be of SMD design with a fixed light spectrum or of multichannel design, where each light color is given a separate channel to control light output from 0-100%. Such a spectrum may then be controlled variably, according to demand. The energy input may be reduced by bringing the LED light source as close as possible to the tube. Further, the energy input may be reduced if the specific light spectrum is adjusted to the particular microorganisms to be cultivated. With an arrangement described in this particular embodiment, there is also no inherent shadowing by the tube arrangement itself.

The light sleeves may be used in different sizes for different tube diameters. The LED light sources may be perfectly tuned to the photosynthesis spectrum of the microorganism cultures, which may be realized, for example, with the aid of various measuring and/or monitoring devices. In order to be able to connect such measuring and/or monitoring devices to the light sleeves, these advantageously have at least one electronic interface.

For example, a fluorescence measurement may be carried out by means of a mini-camera, which may be used to optimally determine the optimum use of light, the harvesting time and other parameters. A temperature sensor may also be used, for example, to measure the optimum temperature for the microorganism culture and, if necessary, actively adjust it via the cooling channels of the LED light array.

1. A light and temperature device for increasing and optimizing biomass production in bioreactors, comprising an LED light arrangement, said LED light arrangement having LED light arrays and cooling fins facing away from said LED light arrays; and at least one fastening means for releasably attaching the light arrangement to a light-transmissive incubation container, wherein the LED light arrangement is aligned with the incubation container such that light radiation emitted from the LED light arrangement is directed towards the incubation container.

2. The light and temperature device according to claim 1, wherein the light and temperature device is formed as a sleeve, said sleeve comprising a shell which is detachably arranged around the incubation container again by means of the fastening means.

3. The light and temperature device according to claim 1 wherein the light and temperature device comprises at least one channel for cooling water or cooling liquid, the channel being arranged in the LED light arrangement.

4. The light and temperature device according to claim 1 wherein the light and temperature device comprises at least one electrical interface for connecting at least one of a measuring device and a monitoring device.

5. The light and temperature device according to claim 4, wherein the at least one of a measuring device and a monitoring device comprise at least one of cameras, CCD cameras, temperature sensors, spectrometers, viscometers, fluorescence/luminescence measuring devices and chlorophyll measuring devices.

6. The light and temperature device according to claim 1 also comprising insulation positioned between and/or laterally of the LED light arrangement and the incubation container and attached in such a way that light irradiation of the LED light arrangement onto the incubation container is not influenced thereby.

7. The light and temperature device according to claim 1 wherein at least one LED light arrangement is arranged around a longitudinal axis of the incubation container in angular steps from 1 degree to 360 degrees.

8. The light and temperature device according to claim 1 wherein the LED light arrangements are configured to emit a fixed light spectrum.

9. The light and temperature device according to claim 1 wherein the light and temperature device is designed for detachable attachment to a tubular, cylindrical, cuboidal, prismatic, pentahedral, hexahedral, heptahedral, octahedral or other geometric incubation container.

10. The light and temperature device according to claim 1 wherein the fastening means is a clamp.

11. The light and temperature device of claim 5 wherein the at least one of a measuring device and a monitoring device are designed for measuring at least one of fluorescence, luminescence, radiation, fluidity, color changes and chlorophyll content.

12. The light and temperature device according to claim 1 also comprising insulation that encases the entire incubation container with the exception of the LED light arrangement.

13. The light and temperature device according to claim 1 wherein at least one LED light arrangement is arranged around a longitudinal axis of the incubation container in angular steps of 90 degrees, 135 degrees or 180 degrees.

14. The light and temperature device according to claim 1 wherein the LED light arrangements are configured to emit a variable light spectrum, each light color having a separate channel for controlling light output from 0%-100%.
